(19) United States
(12) Patent Application Publication
(10) Pub. No.: US 2019/0099191 A1
(43) Pub. Date: Apr. 4, 2019

(54) DISTALLY REAMING BROACH

(71) Applicant: DEPUY SYNTHES PRODUCTS, INC., Raynham, MA (US)

(72) Inventors: DANIEL N. HUFF, WARSAW, IN (US); JUAN A. DACCACH, WARSAW, IN (US)

(21) Appl. No.: 15/720,132

(22) Filed: Sep. 29, 2017

(51) Int. Cl. A61B 17/16; A61B 17/56; A61F 2/28; A61F 2/46

(52) U.S. Cl. CPC A61B 17/1659; A61B 17/1631; A61B 17/164; A61B 2017/00473; A61F 2/28; A61F 2/4644; A61B 17/56

(57) ABSTRACT

An instrument for preparing bone. The instrument includes a proximal broach having an outer surface and a canal extending through the length of the proximal broach. The instrument also includes a distal reamer coupled to the proximal broach such that distal reamer is adapted to rotate relative to the proximal broach.

### TECHNICAL FIELD OF THE INVENTION

The present invention relates generally to the field of orthopedics, and, more particularly, to an improved instrument for enhanced broaching of a cavity in a specific bone before receiving a prosthesis.

### BACKGROUND

For implantation of prosthetic stems, such as femoral hip stems, accurate preparation of the bone or intramedullary canal is important in order to guarantee good contact between the prosthesis stem and the bone. The underlying concept behind adequate preparation is that a precise bone envelope reduces the gaps between the stem and the bone, thereby improving the initial and long-term bone ingrowth/fixation. The bone canal is presently prepared for implantation of a prosthetic stem by broaching and reaming a resected end of a bone, such as the proximal femur, and then preparing an area adjacent to the prepared cavity in order to provide a seat for the prosthetic stem or a proximal sleeve coupled to the stem of a modular prosthetic system.

Preparation of the bone cavity can be achieved using tools such as reamers and broaches. A reamer is a rotating tool which can be used to create a cavity with a circular cross-section, which will frequently be constant along its length. A broach, such as disclosed in U.S. Pat. No. 5,089,004, is generally manipulated in an axial direction. It can be used to create a cavity with a noncircular cross-section. It is suitable for creation of the portion of a medullary cavity that is to receive the tapered portion of a prosthesis, in particular to ensure that the tapered shape of the prosthesis, which can be a complicated irregular shape, is properly matched by the shape of the internal surface of the bone cavity.

In current surgeries, switching between broaches and reamers can take up valuable operating time. Also, one current need is reducing thigh pain, which may be enhanced by inadequate broaching and reaming. Therefore, the present invention seeks to alleviate these issues.

### SUMMARY OF THE INVENTION

According to one embodiment of the present invention, an instrument for preparing a bone is provided. The instrument includes a proximal broach having an outer surface and a canal extending through the length of the proximal broach. The instrument further includes a distal reamer coupled to the proximal broach such that the distal reamer is adapted to rotate relative to the proximal broach.

According to another embodiment of the present invention, a method of cutting bone is provided. The method includes impacting a reaming broach into the bone, the reaming broach having an outer surface and a canal extending through the length of the proximal broach and a distal reamer coupled to the proximal broach. Once the reaming broach is fully seated in the bone, the method further includes rotating the distal reamer relative to the proximal broach.

According to yet another embodiment of the present invention, a method for cutting a bone is provided. The method includes assembling a distal reamer to a proximal broach. The proximal broach has an outer surface and a canal extending through the length of the proximal broach. The method further includes impacting the assembled distal reamer and proximal broach into the bone and then rotating the distal reamer relative to the proximal broach.

### BRIEF DESCRIPTION OF THE DRAWINGS

FIG. 1 is a medial view of a reaming broach according to one aspect of the invention.

FIG. 2 is a medial view of the proximal broach of FIG. 1.

FIG. 3 is a medial view of the distal reamer on the reaming broach of FIG. 1.

FIG. 4 is a view of the reaming broach of FIG. 1 as shown in bone.

FIG. 5 is a flowchart describing one method of using the reaming broach.

FIG. 6 is a view of the reaming broach of FIG. 1 attached to a handle.

### DETAILED DESCRIPTION

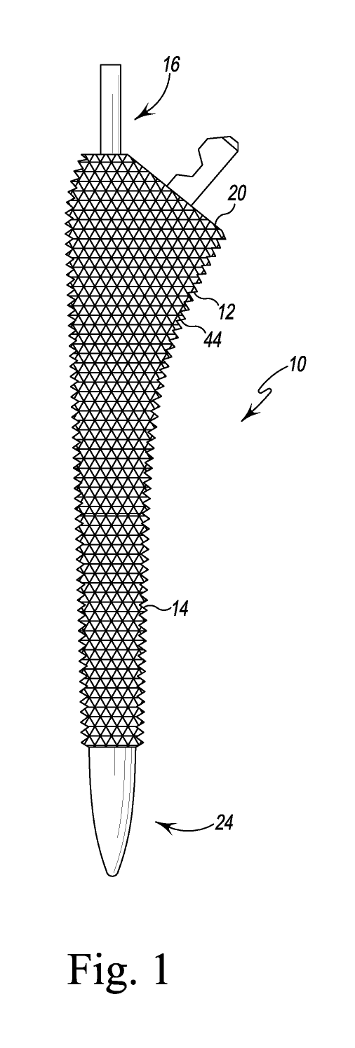

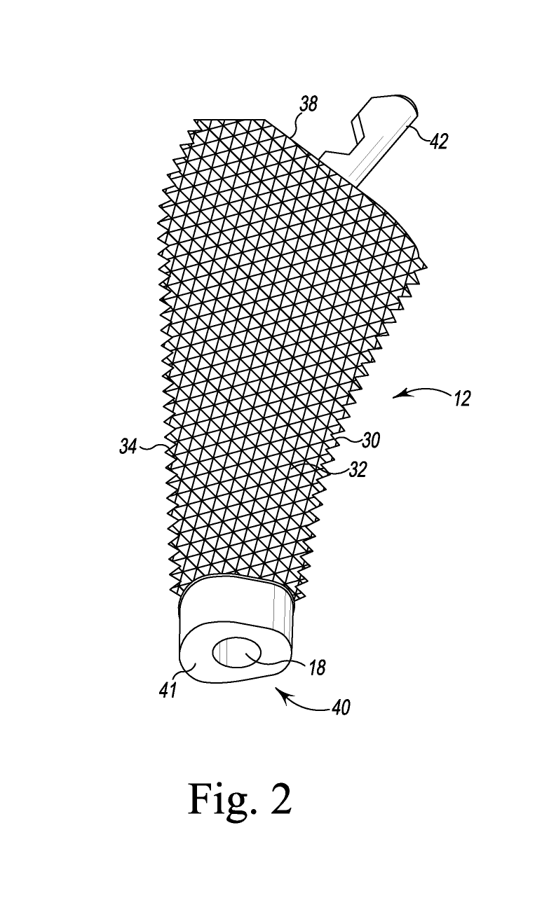

Like reference numerals refer to like parts throughout the following description and the accompanying drawings. Turning now to FIG. 1, a reaming broach 10 according to one embodiment of the present invention is illustrated. The reaming broach 10 includes a proximal broach 12 and a distal reamer 14. A drive shaft 16 extends through a canal 18 (shown in FIG. 2) of the proximal broach 12 and attaches to the distal reamer 14. As shown, both the distal reamer 14 and the proximal broach 12 include cutting teeth 20, 22, respectively. At the distal most portion 24 of the reamer 14, the distal reamer may be smooth, without any teeth. In other embodiments, the teeth 22 of the distal reamer may extend throughout the length of the reamer 14.

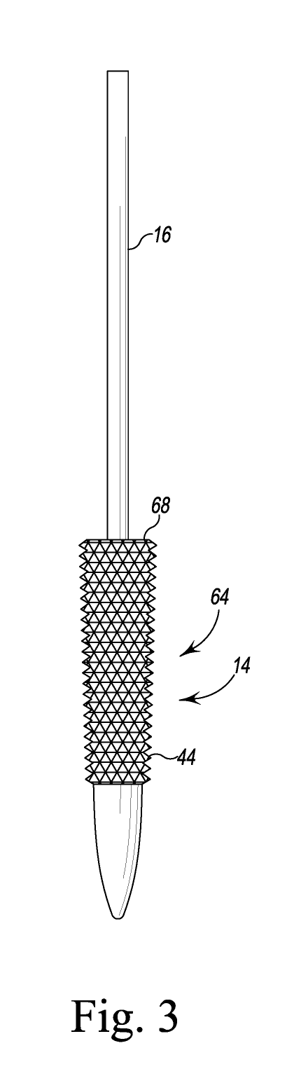

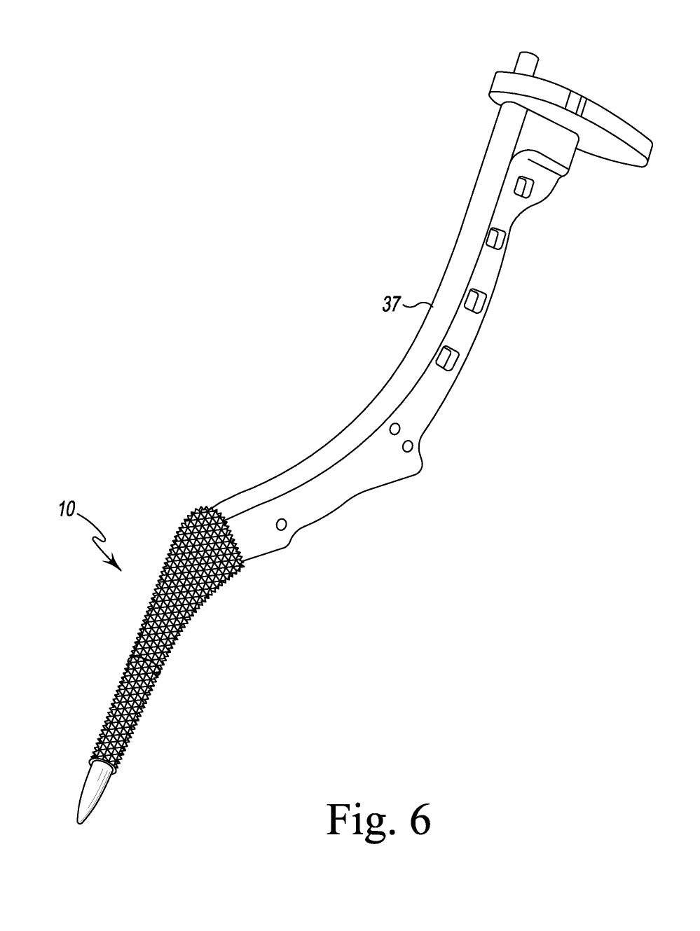

Turning now to FIG. 2, the proximal broach 12 will be described in more detail. The proximal broach 12 has an outer surface 26 that is the same general shape as the configuration as an implant (not shown). In the illustrated embodiment, the proximal broach is in the shape of the proximal portion of a femoral stem. Other shapes (such as those for humeral stems) are also contemplated. The broach 12 includes a medial side 30, a lateral side 34, an anterior side 32, and a posterior side 36. The broach 12 also includes a proximal portion 38 and a distal portion 40. Located at the top of the proximal portion 38 is a handle connection 42. The handle connection 42 allows the broach 12 to be coupled to a handle (37 of FIG. 6) or driver (not shown). As mentioned above, the canal 18 extends from the proximal portion 38 of the broach 12 through the length of the broach to the distal portion 40. The distal portion 40 includes a flat end 41, that will abut a flat end 68 (FIG. 3) of the reamer 14.

The proximal broach 12 has cutting teeth 44 extending all around the outer surface 26. In the shown embodiment, the teeth are all “extraction” or “cutting” teeth, meaning they are able to cut into the bone. In other embodiments, “compaction” teeth (teeth which compact the bone as opposed to cutting into it) or a combination between extraction and cutting teeth may be used.

Turning now to FIG. 3, a view of the reamer 14 is shown. As shown, the reamer includes the drive shaft 16 that extends from a proximal portion 62 toward a distal portion 64. The distal portion 64 is a reamer including a plurality of cutting teeth 66. In the illustrated embodiment, the teeth are “extraction teeth” and are in the same pattern as along the proximal broach 12. In other embodiments, the teeth 66 of the distal portion 64 may be different than the teeth 44 of the proximal broach. The teeth 66 may extend all the way to the tip of the reamer 14, or, as stated above, the tip may be smooth. When assembled as shown in FIG. 1, the end 41 of the distal broach 12 abuts a flat surface 68 on the reamer 14. The teeth 66 of the reamer and the teeth 44 of the broach are similar, so that when the reaming broach 10 is being used as a broach, there is no break or discontinuity of the teeth.

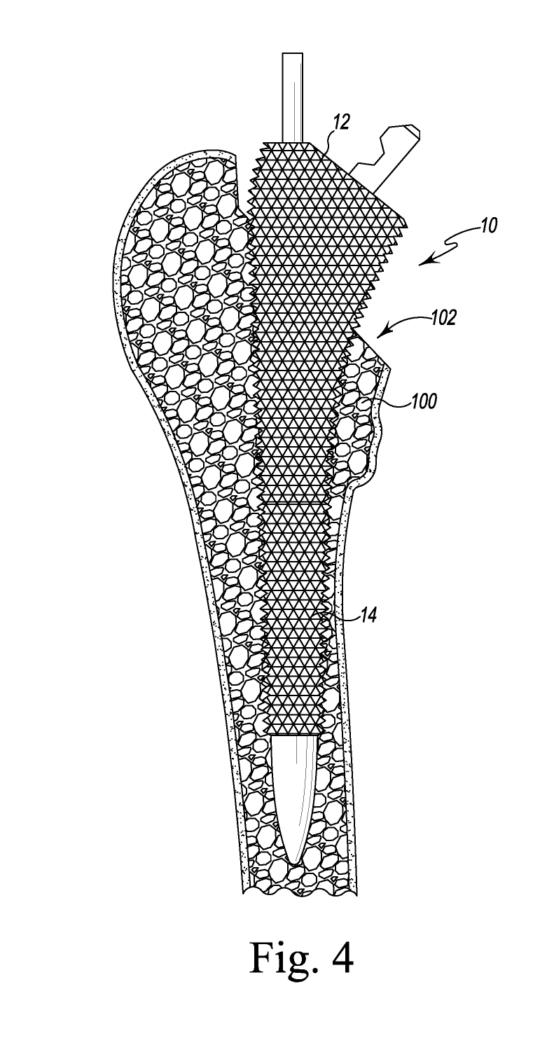

FIG. 4 illustrates the reaming broach 10 in a femur 100. The head of the femur 100 has been resected from the proximal end of the femur. The reaming broach 10 is inserted into an intramedullary canal 102 of the femur 100. Cancellous bone, which is softer and somewhat spongy, surrounds the intramedullary canal. Surrounding the cancellous bone is cortical bone, which is stronger. The proximal broach 12 is used to cut into the bone in the proximal portion of the femur 100. Once the broach 12 is firmly seated, then the user can activate the distal reamer 14 to further cut the bone in the distal portion of the femur 100 without extracting the reaming broach.

In one embodiment, the broaches are made entirely from stainless steel. Alternatively, other metals may be used. Additionally, in some embodiments, the broaches may be made with a hard plastic, such as PEEK.

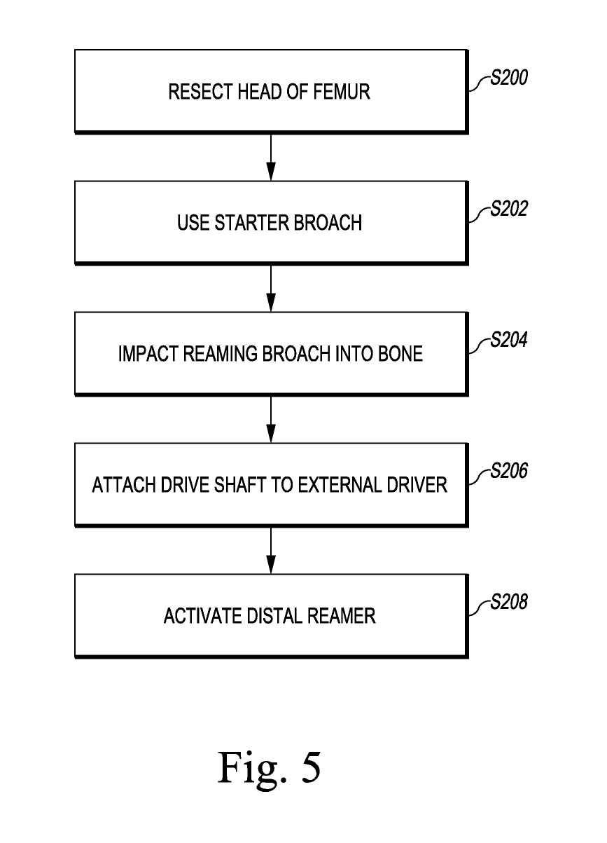

Turning now to FIG. 5, a flow chart of the method of using the reaming broach will be described. At step s200, the head of the femur 100 is resected. Next, a starter broach may be used at step s202. In other embodiments, the user may start directly with a reaming broach. At step s204, a first size of the reaming broach 10 is impacted into the femur 100 by hitting a strike plate (shown in FIG. 6) to hammer the reaming broach into the bone. Once the proximal broach 12 is firmly seated in the bone, then the user may attach the drive shaft 16 to an external driver (not shown) at step s206. The driver, once actuated will cause the distal reamer 14 to spin, reaming out the distal portion of the bone. The proximal broach 12, however, will stay in place. By keeping the proximal broach 12 in place, the proximal broach can allow proper guidance to the distal reamer 14 without extraction of the complete instrument. This may reduce need for extra instruments and could enhance the fluidity of the surgical procedure.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

What is claimed is:

1. An instrument for preparing bone, the instrument comprising:
a proximal broach having an outer surface and a canal extending through the length of the proximal broach; and
a distal reamer coupled to the proximal broach such that distal reamer is adapted to rotate relative to the proximal broach.

2. The instrument of claim 1, wherein the proximal broach has a plurality of teeth extending around the outer surface.

3. The instrument of claim 1, further comprising a shaft coupled to the distal reamer, such that when the shaft rotates, the distal reamer rotates.

4. The instrument of claim 3, wherein the shaft and the distal reamer are a single piece.

5. The instrument of claim 3, wherein the shaft extends through the canal of the proximal broach.

6. The instrument of claim 1, wherein the proximal broach further includes a handle connection adapted to be coupled to a handle.

7. The instrument of claim 1, wherein the proximal broach includes a flat end and the distal reamer includes a flat surface such that when the proximal broach and distal reamer are assembled, the flat end of the broach abuts the flat surface of the reamer.

8. The instrument of claim 1, wherein the broach includes a plurality of teeth and the reamer includes a plurality of teeth.

9. The instrument of claim 1, wherein both the teeth of the broach and reamer are extraction teeth.

10. The instrument of claim 8, wherein both the teeth of the distal reamer and the proximal broach are arranged in a pattern and the pattern is continuous across the broach and the reamer when assembled.

11. A method of cutting a bone comprising:
impacting a reaming broach into the bone, the reaming broach including a proximal broach having an outer surface and a canal extending through a length of the proximal broach and a distal reamer coupled to the proximal broach;
once the reaming broach is fully seated in the bone, rotating the distal reamer relative to the proximal broach.

12. The method of claim 11, further comprising resecting a head of the bone prior to impacting the reaming broach.

13. The method of claim 11, wherein the distal reamer includes a shaft, such that when the distal reamer is coupled to the proximal broach, the shaft extends through the canal of the proximal broach.

14. The method of claim 13, further comprising attaching the shaft to an external driver.

15. The method of claim 13, wherein rotating the distal reamer includes activating the external driver, causing the distal reamer to rotate.

16. A method for cutting a bone comprising:
assembling a distal reamer to a proximal broach, having an outer surface and a canal extending through a length of the proximal broach;
impacting the assembled distal reamer and proximal broach into a bone;
rotating the distal reamer relative to the proximal broach.

17. The method of claim 16, wherein the distal reamer includes a shaft and assembling the distal reamer to the proximal broach includes inserting the shaft through the canal on the broach.

18. The method of claim 17, further comprising attaching the shaft to an external driver.

19. The method of claim 18, wherein rotating the distal reamer includes activating the external driver.

20. The method of claim 16, wherein the broach includes a handle.
